(19) United States
(12) Patent Application Publication
(10) Pub. No.: US 2023/0113817 A1
(43) Pub. Date: Apr. 13, 2023

(54) DILEUCINE COMPOSITIONS AND METHODS OF USE THEREOF FOR FAT LOSS

(71) Applicant: Ingenious Ingredients, LP, Lewisville, TX (US)

(72) Inventors: Ralf Jäger, Whitefish Bay, WI (US); Shawn D. Wells, Frisco, TX (US); Kylin Liao, Plano, TX (US); Martin Purpura, Spring, TX (US)

(21) Appl. No.: 17/964,903

(22) Filed: Oct. 12, 2022

Related U.S. Application Data
(60) Provisional application No. 63/254,909, filed on Oct. 12, 2021.

(51) Int. Cl. A61K 38/05; A61K 31/522; A61K 36/82; A61K 31/205; A61K 31/165; A61K 36/258; A61K 31/145; A61K 31/045; A61K 35/64; A61K 36/38; A61K 36/752; A61K 31/475; A61K 36/48; A61K 31/736; A61K 36/24; A61K 31/405; A61K 31/201; A61K 36/185; A61K 38/06; A61P 3/04

(52) U.S. Cl. CPC A61K 38/05; A61K 31/522; A61K 36/82; A61K 31/205; A61K 31/165; A61K 36/258; A61K 31/145; A61K 31/045; A61K 35/64; A61K 36/38; A61K 36/752; A61K 31/475; A61K 36/48; A61K 31/736; A61K 36/24; A61K 31/405; A61K 31/201; A61K 36/185; A61K 38/06; A61P 3/04

(57) ABSTRACT

Described herein are compositions for reducing body weight, reducing body fat, improving body composition and/or preventing obesity and related conditions. In certain aspects, the compositions comprise dileucine, and a pharmaceutically acceptable carrier thereof. In certain embodiments, the composition comprises dileucine and leucine. In further aspects, the dileucine is present from about 10% to 90% (w/w). In further aspects, the dileucine is present from about 20% to 80% (w/w). In further aspects, wherein the dileucine is present from about 30% to 70% (w/w). In further aspects, the dileucine is present from about 40% to 60% (w/w).

### CROSS-REFERENCE TO RELATED APPLICATION(S)

This application claims the benefit under 35 U.S.C. § 119(e) to U.S. Provisional Application 63/254,909, filed Oct. 12, 2021, and entitled “DILEUCINE COMPOSITIONS AND METHODS OF USE THEREOF FOR WEIGHT LOSS,” which is hereby incorporated herein by reference in its entirety for all purposes.

### BACKGROUND

Obesity is a significant problem in the Western world, with estimates of its prevalence ranging from 30% to 50% of the middle-aged population. The number of overweight (defined as a person with a body mass index (BMI) equal to or greater than 25 kg/m²) and obese (defined as a person with a BMI equal to or greater than 30 kg/m²) Americans has continued to increase since 1960, a trend that is not slowing down. Today, approximately 64.5% of adult Americans (about 199 million) are categorized as being overweight or obese. Obesity is becoming a growing concern as the number of people with obesity continues to increase and more is learned about the negative health effects of obesity. Obesity can lead to type 2 diabetes, heart disease, and some cancers. Each year, obesity causes at least 300,000 deaths in the U.S., and healthcare costs of American adults with obesity amount to more than $147 billion. There is a need in the art for compositions and methods to more effectively achieve weight loss.

### BRIEF SUMMARY

Described herein are compositions for reducing body weight, reducing body fat, improving body composition and/or preventing obesity and related conditions. In certain aspects, the compositions comprise dileucine, and a pharmaceutically acceptable carrier thereof. In certain embodiments, the composition comprises dileucine and leucine. In further aspects, the dileucine is present from about 10% to 90% (w/w). In further aspects, the dileucine is present from about 20% to 80% (w/w). In further aspects, wherein the dileucine is present from about 30% to 70% (w/w). In further aspects, the dileucine is present from about 40% to 60% (w/w). In still further aspects, the dileucine is present from about 50% (w/w).

Described herein are various embodiments relating to compositions of and methods reducing body fat in a subject in need thereof. In certain aspects, disclosed is a method for increasing weight loss a subject, by administering to the subject an effective amount of a composition that comprises at least one amino acid or peptide chosen from: di-leucine, tri-leucine, and Leu-Leu-R, wherein R is an amino acid or an amino acid derivative, and pharmaceutically acceptable salts thereof. In certain aspects, the compound is di-leucine. In certain aspects, the at least one amino acid or peptide comprises leucine and di-leucine. In further aspects the dileucine is present from about 10% to about 90% (w/w). In further aspects, the dileucine is present from about 30% to about 70% (w/w). In still further aspects, the dileucine is present at about 50% (w/w).

In further aspects, the composition of a salt of di-leucine acetate. In certain aspects, the composition is Leu-Leu-R and R is a branched-chain amino acid. In further aspects, R is an essential amino acid. In yet further aspects, R is a conditionally essential amino selected from the group consisting of arginine, cysteine, glutamine, glycine, proline, and tyrosine. In exemplary embodiments, R is tyrosine. According to certain alternative embodiments, R is a non-essential amino selected from the group consisting of: alanine, aspartic acid, asparagine, glutamic acid, serine, selenocysteine and pyrrolysine. In further alternative embodiments, R is an amino acid derivative selected from a list consisting of: creatine, carnitine, creatinol, beta-alanine, taurine, and beta-hydroxy beta-methylbutyrate.

In certain aspects, the administration of the composition to the subject synergistically increases weight loss and/or lipolysis relative to administration of a composition comprising leucine without dileucine.

Further disclosed herein is a method for preventing or treating obesity in a subject comprising administering to the subject an effective amount of a composition that comprises at least one amino acid or peptide chosen from: di-leucine, tri-leucine, and Leu-Leu-R, wherein R is an amino acid or an amino acid derivative. In certain aspects the compositions comprises leucine and dileucine. In certain aspects, the method is used to treat or prevent obesity that is associated with metabolic syndrome.

In some aspects, the dileucine is present from about 10% to 90% (w/w). In further aspects, the dileucine is present from about 30% to 70% (w/w). In further aspects, the dileucine is present at about 50% (w/w).

In various aspects, the administration of the composition to the subject synergistically increases the plasma levels of leucine relative to administration of a composition comprising leucine without dileucine. In certain aspects the composition is administered in a therapeutically effective amount.

Further disclosed herein are compositions for the treatment of conditions comprising dileucine, leucine, and pharmaceutically acceptable carriers thereof. In certain aspects the condition is at least one of obesity, immune system function associated disorders, insulin secretion associated disorders, diabetes, virulence associated conditions, cardiovascular disorder, cardiac disorders, degenerative diseases, sarcopenia, ocular disease, fibrotic diseases, aging associated disorders, improving skin hydration and collagen synthesis, liver disease, Crohn's disease.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 shows exemplary data from strength training studies, according to certain embodiments.

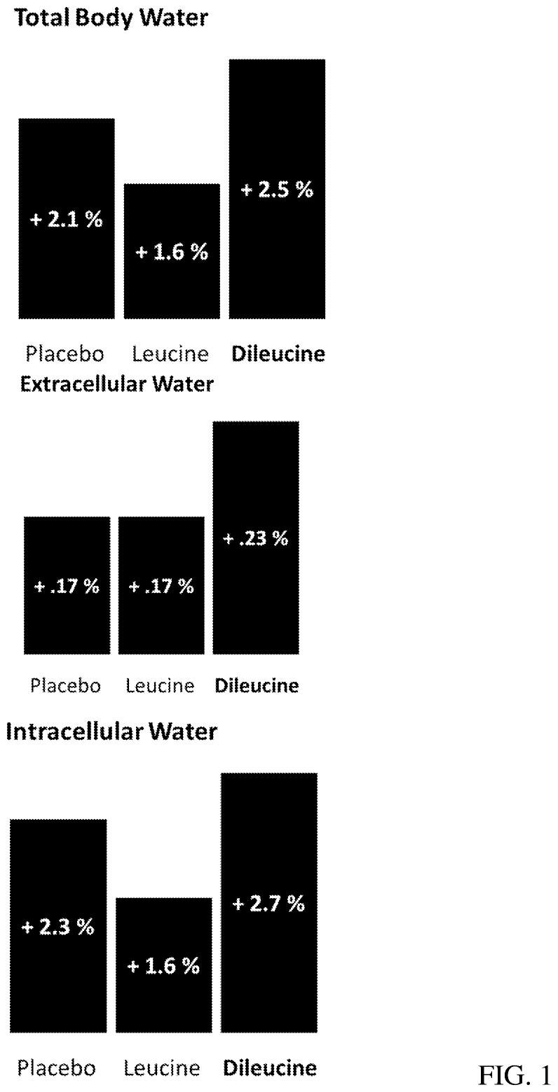

FIG. 2 shows exemplary data from strength training studies, according to certain embodiments.

FIG. 3 shows exemplary data from strength training studies, according to certain embodiments.

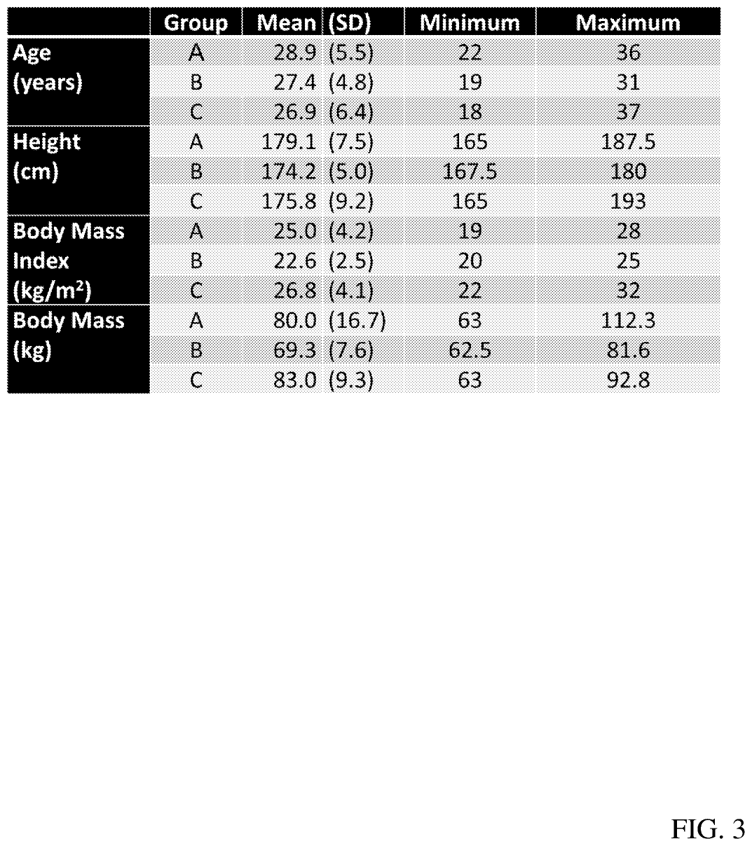

### DETAILED DESCRIPTION

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

As used herein, the term “subject” refers to the target of administration, e.g. a subject. Thus the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more body weight disorders prior to the administering step. In some aspects of the disclosed method, the subject has been diagnosed with a need for increasing weight loss prior to the administering step.

The term “overweight” is defined as the condition wherein the individual has a BMI greater than or 25 kg/m² and less than 30 kg/m². The terms “overweight” and “pre-obese” are used interchangeably.

As used herein, the term “obesity” is defined as the condition wherein the individual has a BMI equal to or greater than 30 kg/m². According to a WHO definition the term obesity may be categorized as follows: the term “class I obesity” is the condition wherein the BMI is equal to or greater than 30 kg/m² but lower than 35 kg/m²; the term “class II obesity” is the condition wherein the BMI is equal to or greater than 35 kg/m² but lower than 40 kg/m²; the term “class III obesity” is the condition wherein the BMI is equal to or greater than 40 kg/m².

As used herein, the terms “manage,” “managing,” and “management” encompass preventing, delaying, or reducing the severity of a recurrence of an adipose associated body composition or body weight disorder, such as obesity, lipodystrophy, diabetes or metabolic syndrome, fibrosis and cancer in a patient who has already suffered from such a disease, disorder or condition. The terms encompass modulating the threshold, development, and/or duration of the adipose associated body composition or body weight disorder, such as obesity, lipodystrophy, diabetes or metabolic syndrome, fibrosis and cancer or changing how a patient responds to the adipose associated body composition or body weight disorder.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with obesity” means having been subjected to a physical examination by a person of skill, for example, a physician, and found to have a condition that can be diagnosed or treated by a compound or composition that can reduce body mass. As a further example, “diagnosed with a need for weight loss” refers to having been subjected to a physical examination by a person of skill, for example, a physician, and found to have a condition characterized by excess of body fat or other disease wherein decreasing body fat would be beneficial to the subject. Such a diagnosis can be in reference to a disorder, such as obesity, metabolic syndrome, and the like, as discussed herein.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to excess adipose tissue) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles would either completely lack particles, or so nearly completely lack particles that the effect would be the same as if it completely lacked particles. In other words, a composition that is “substantially free of an ingredient or element may still actually contain such item as long as there is no measurable effect thereof.

As used herein, the term “synergistic effect” or grammatical variations thereof means and includes a cooperative action encountered in a combination of two or more active compounds in which the combined activity of the two or more active compounds exceeds the sum of the activity of each active compound alone. The term “synergistically effective amount,” as used herein, means and includes an amount of two or more active compounds that provides a synergistic effect defined above.

Disclosed herein are compositions and methods for promoting weight loss and/or preventing or obesity. In certain aspects, the disclosed method comprises administering a composition to a subject where the composition that comprises the compound di-leucine. Di-leucine refers to a dipeptide comprised of two L-leucines. In certain aspects, di-leucine can have the structure:

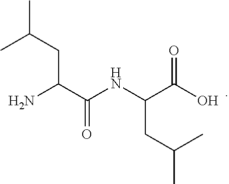 [SMILES: CC(C)CC(N)C(=O)NC(CC(C)C)C(=O)O]

Di-leucine may also be referred to as L-Leucyl-L-leucine or Leu-Leu and has number CAS #3303-31-9.

In certain aspects, disclosed are compositions for reducing body weight comprising dileucine, leucine, and a pharmaceutically acceptable carrier thereof. In various aspects dileucine is present from about 10% to 90% (w/w). In further aspects, the dileucine is present from about 20% to 80% (w/w). In further aspects, wherein dileucine is present from about 30% to 70% (w/w). In further aspects, dileucine is present from about 40% to 60% (w/w). In still further aspects, dileucine is present from about 50% (w/w).

In alternative embodiments, dileucine is present from about 10%-90% (w/w) and leucine is present from about 90%-10% (w/w). In further embodiments, dileucine is present from about 20%-80% (w/w) and leucine is present from about 80%-20% (w/w). In further embodiments, dileucine is present from about 30%-70% (w/w) and leucine is present from about 70%-30% (w/w). In further embodiments, dileucine is present from about 40%-60% (w/w) and leucine is present from about 60%-40% (w/w). In further embodiments, dileucine is present at about 50% (w/w) and leucine is present at about 50% (w/w).

In certain aspects, the disclosed method comprises administering a composition comprising di-leucine salt. In exemplary embodiments, the composition is a di-leucine acetate salt, which may have the structure:

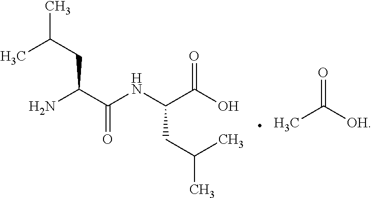

In certain aspects, the disclosed method comprises administering a composition to a subject where the composition comprises tri-leucine. Trileucine means a tripeptide comprising of three L-leucines. In certain aspects, tri-leucine has the structure:

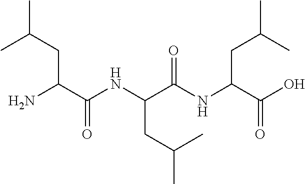 [SMILES: CC(C)CC(N)C(=O)NC(CC(C)C)C(=O)NC(CC(C)C)C(=O)O]

Tri-leucine may also be referred to as TRILEUCINE;LEU-LEU-LEU;H-LEU-LEU-LEU-OH;L-LEUCYLLEUCYLLEUCINE;leucyl-leucyl-leucine;Leu-leu-leucrystalline;L-LEUCY-L-LEUCYL-L-LEUCINE;L-LEUCYL-L-LEUCYL-L-LEUCINE;Leu-Leu-Leu-OH≥S)-2-((S)-2-((S)-2-Amino-4-methylpentanamido)-4-methylpentanamido)-4-methylpentanoic acid.

In further aspects, the disclosed method comprises administering a composition to a subject where the composition comprises tripeptide comprising two L-Leucine units and one amino acid or amino acid derivative. According to certain embodiments, the amino acid is selected from a group of branched-chain amino acids (BCAA), including, but not limited to, isoleucine, leucine, and valine. In further embodiments, the amino acid is selected from the group of essential amino acids, including, but not limited to, histidine, isoleucine, leucine, lysine, methionine, phenylalanine, threonine, tryptophan, and valine. In still further embodiments, the amino acid is selected from the group of conditionally essential amino acids including, but not limited to, arginine, cysteine, glutamine, glycine, proline, and tyrosine. According to certain embodiments, the conditionally essential amino acid is tyrosine. In exemplary embodiments, the composition comprises a di-leucine tyrosine that has the structure:

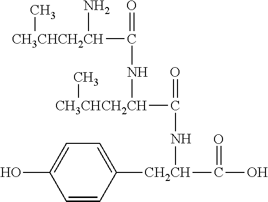

In still further embodiments, the amino acid is selected from the group of non-essential amino acids including, but not limited to, alanine, aspartic acid, asparagine, glutamic acid, serine, selenocysteine and pyrrolysine. In yet further embodiments, the amino acid derivative is selected from the group of creatine, carnitine, creatinol, beta-alanine, taurine, and beta-hydroxy beta-methylbutyrate.

In certain aspects, disclosed is a method for reducing body weight and/or body fat in a subject comprising administering to the subject an effective amount of a composition that comprises at least one amino acid or peptide chosen from: di-leucine, tri-leucine, and Leu-Leu-R, wherein R—is an amino acid or an amino acid derivative, and pharmaceutically acceptable salts thereof. In various aspects the at least one amino acid or peptide comprises leucine and di-leucine and/or and pharmaceutically acceptable salts thereof.

In certain embodiments, dileucine is present from about 10% (w/w) to about 90% (w/w). In further embodiments, dileucine is present from about 30% to 70% (w/w). In further embodiments, dileucine is present at about 50% (w/w).

In various alternative embodiments, dileucine is present from about 10%-90% (w/w) and leucine is present from about 90%-10% (w/w). In further embodiments, dileucine is present from about 30%-70% (w/w) and leucine is present from about 70%-30% (w/w). In further embodiments, dileucine is present at about 50% (w/w) and leucine is present at about 50% (w/w).

In these and other embodiments, the administration of the composition to the subject synergistically increases the plasma levels of leucine relative to administration of a composition comprising leucine without dileucine. Additionally, the administration of the composition to the subject synergistically reducing body weight and/or body fat relative to administration of a composition comprising leucine without dileucine.

In various aspects the compositions comprise at least about 95% dileucine; and between about 0.1%-5% tri-leucine, and pharmaceutically acceptable salts thereof. In further aspects, the tri-leucine is present at an amount between about 0.1%-3% and the composition further comprising about 0.1%-2% tetra-leucine. In yet further aspects, the tri-leucine is present at an amount of about 0.4% and the tetra-leucine is present at an amount of about 0.2%.

According to certain alternative embodiments, the composition comprises at least about 95% dileucine; and between about 0.1%-5% tetra-leucine, and pharmaceutically acceptable salts thereof.

In certain aspects of the foregoing embodiments, the composition is substantially free of leucine.

According to certain further aspects, disclosed is a method for reducing body weight of a subject, the method comprising administering to the subject an effective amount of a composition comprising at least about 95% dileucine; and between about 0.1%-5% leucine, and pharmaceutically acceptable salts thereof.

In certain aspects, the compositions administered according to the disclosed methods are produced through bacterial fermentation. According to these embodiments, fermentation techniques are employed utilizing di-/tri-/tetra-peptide-forming enzymes that directly links amino acids, followed by extraction processes.

In certain aspects, disclosed herein are methods to promote weight loss through the administration of an effective amount of one or more compositions disclosed herein. According to certain aspects, administration of effective amounts of the disclosed compositions are used in treating diabetes mellitus; preventing, slowing progression of, delaying or treating of a condition or disorder selected from the group consisting of complications of diabetes mellitus; preventing, slowing the progression of, delaying or treating a metabolic disorder selected from the group consisting of type 1 diabetes mellitus, type 2 diabetes mellitus, impaired glucose tolerance (IGT), impaired fasting blood glucose (IFG), hyperglycemia, postprandial hyperglycemia, overweight, obesity, metabolic syndrome and gestational diabetes; or improving glycemic control and/or for reducing of fasting plasma glucose, of postprandial plasma glucose and/or of glycosylated hemoglobin HbA1c; or preventing, slowing, delaying or reversing progression from impaired glucose tolerance IGT), impaired fasting blood glucose (IFG), insulin resistance and/or from metabolic syndrome to type 2 diabetes mellitus; or preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischemia, arteriosclerosis, myocardial infarction, stroke and peripheral arterial occlusive disease; or reducing body weight and/or body fat or preventing an increase in body weight and/or body fat or facilitating a reduction in body weight and/or body fat; or preventing, slowing, delaying or treating diseases or conditions attributed to an abnormal accumulation of ectopic fat; or maintaining and/or improving the insulin sensitivity and/or for treating or preventing hyperinsulinemia and/or insulin resistance; preventing, slowing progression of, delaying, or treating new onset diabetes after transplantation (NODAT) and/or post-transplant metabolic syndrome (PTMS); preventing, delaying, or reducing NODAT and/or PTMS associated complications including micro- and macrovascular diseases and events, graft rejection, infection, and death; treating diabetes associated with cystic fibrosis treating hyperuricemia and hyperuricemia associated conditions; treating or prevention kidney stones; treating hyponatremia; in a patient in need thereof.

Another aspect encompasses a combination therapy to regulate fat storage, energy utilization, and/or weight loss in a subject. In an exemplary embodiment, a combination for increasing energy utilization, decreasing body fat or for promoting weight loss may include combining the methods and compositions disclosed with a procedure or therapy such as a pharmaceutical therapy, gastric bypass, duodenojejunal bypass, biliopancreatic diversion, vertical sleeve gastrectomy, adjustable gastric banding, vertical banded gastroplasty, intragastric balloon therapy, gastric plication, Magenstrasse and Mill, small bowel transposition, biliary diversion, brown adipose tissue modulation (e.g., controlled activation, enhanced differentiation, supplemental implantation, etc.), pharmaceutical administration, electrical stimulation of nerves that innervate at least a portion of the gastrointestinal tract, therapies impacting circadian rhythms, bile acid modulation, intestinal mucus production and metabolism, duodenal endoluminal barrier or similar manipulations of the gastrointestinal tract. For example, a composition dileucine can be administered to the subject prior to, concurrently with or after a gastric bypass or other gastrointestinal or bariatric procedure.

In certain aspects, administration of the disclosed compositions is effective at preventing reducing body weight and/or body fat or preventing an increase in body weight and/or body fat or facilitating a reduction in body weight and/or body fat; or preventing, slowing, delaying or treating diseases or conditions attributed to an abnormal accumulation of liver fat; or maintaining and/or improving the insulin sensitivity and/or for treating or preventing hyperinsulinemia and/or insulin resistance.

Disclosed herein is a method for prevention or treating muscle atrophy in a subject, the method comprises administering to the subject an effective amount of a disclosed composition. In certain aspects the composition comprises leucine and dileucine.

In various embodiments, dileucine is present from about 10% (w/w) to about 90% (w/w). In further embodiments, dileucine is present from about 30% to 70% (w/w). In further embodiments, dileucine is present at about 50% (w/w).

In various alternative embodiments, dileucine is present from about 10%-90% (w/w) and leucine is present from about 90%-10% (w/w) of the combined weight of leucine and dileucine. In further embodiments, dileucine is present from about 30%-70% (w/w) and leucine is present from about 70%-30% (w/w) of the combined weight of leucine and dileucine. In further embodiments, dileucine is present at about 50% (w/w) and leucine is present at about 50% (w/w) of the combined weight of leucine and dileucine.

According to certain embodiments, fat loss is promoted through inducing thermogenesis in the subject. According to exemplary implementations of these embodiments, the composition may also include one or more compounds selected from: caffeine, green tea, capsaicin, Garcinia cambogia, yohimbine and bitter orange.

According to further embodiments, fat loss is promoted through suppression of appetite in the subject. In exemplary implementations of these embodiments, the composition further may include one or more compounds selected from: fenugreek, glucomannan, Gymnema sylvestre, 5-HTP, Caralluma fimbriata, green tea extract, Conjugated linoleic acid, Garcinia cambogia, and Yerba mate.

According to still further embodiments, fat loss is promoted through enhancing lipolysis in the subject. In exemplary implementations of these embodiments, the composition further may include one or more compounds selected from caffeine, green tea extract, L-carnitine, Garcinia cambogia (hydroxycitric acid), capsaicin, ginseng, taurine, silk peptides and octacosanol.

According to certain implementations, the disclosed method further comprises restricting calorie intake of the subject. In exemplary implementations, the amount of fat loss in the subject is greater than that for a subject with an equivalent calorie restriction that has not been provided the composition. According to further implementations, the ratio of fat loss to muscle loss in the subject the subject is greater than that for a subject with an equivalent calorie restriction that has not been provided the composition.

According to certain embodiments of the disclosed method, the composition is administered in a therapeutically effective amount. In further embodiments, the composition is administered in a prophylactically effective amount.

In some embodiments, contemplated methods may further comprise assessing one or more indices of on-going weight loss, e.g. the ketone body production level in a patient; and optionally adjusting the amount administered; thereby optimizing the therapeutic efficacy of the dileucine composition.

According to certain embodiments, administration of the disclosed compositions can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result, for example a weight loss target, is achieved. For example, when about loss of about 20% body weight, about 30% body weight or more has been achieved. A treatment regimen can include a corrective phase, during which a composition dose sufficient to provide reduction of excess adiposity is administered, followed by a maintenance phase, during which a lower dileucine composition dose sufficient to prevent re-development of excess adiposity is administered.

In a further aspect, the disclosed compounds reduce body weight and/or body fat when administered at an oral dose of greater than about 25 mg per day in a human (e.g. the composition is administered such that the subject receives a dose of at least 25 mg per day). In a yet further aspect, the disclosed compounds reduce body weight and/or body fat when administered at an oral dose of greater than about 100 mg per day in a human In a yet further aspect, the disclosed compounds reduce body weight and/or body fat when administered at an oral dose of greater than about 300 mg per day in a human. In a still further aspect, the disclosed compounds reduce body weight and/or body fat when administered at an oral dose of greater than about 400 mg per day in a human. In an even further aspect, the disclosed compounds reduce body weight and/or body fat when administered at an oral dose of greater than about 500 mg per day in a human. In a further aspect, the disclosed compounds reduce body weight and/or body fat when administered at an oral dose of greater than about 750 mg per day in a human. In a yet further aspect, the disclosed compounds reduce body weight and/or body fat when administered at an oral dose of greater than about 1000 mg per day in a human. In a still further aspect, the disclosed compounds reduce body weight and/or body fat when administered at an oral dose of greater than about 2000 mg per day in a human. In an even further aspect, the disclosed compounds reduce body weight and/or body fat when administered at an oral dose of greater than about 3000 mg per day in a human. In a yet further aspect, the disclosed compounds reduce body weight and/or body fat when administered at an oral dose of greater than about 5000 mg per day in a human.

Further disclosed herein is a method for promoting weight management in a subject in need thereof comprising administering to the subject a composition comprising and effective amount of dileucine and paraxanthine. In certain embodiments, paraxanthine is administered to the subject in an amount of between about 50 mg and about 800 mg per day. In further embodiments, between 100 mg and 400 mg per day of paraxanthine are administered. In certain embodiments, the administration of the composition to the subject synergistically reduces body weight and/or body fat relative to administration of a composition comprising dileucine without paraxanthine or paraxanthine without dileucine.

### Nutritional Supplements

The compositions of the disclosure may take the form of dietary supplements or may themselves be used in combination with dietary supplements, also referred to herein as food supplements.

Nutritional supplements may be found in many forms such as tablets, capsules, soft gels, gel caps, liquids, or powders. Some dietary supplements can help ensure an adequate dietary intake of essential nutrients; others may help reduce risk of disease.

### Food Products

The compositions of the disclosure may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the disclosure may take the form of one of the following: A fruit juice; a beverage comprising whey protein: a health or herbal tea, a cocoa drink, a coffee drink, a yoghurt and/or a drinking yoghurt, a cheese, an ice cream, a desserts, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, a calcium fortified soy milk, or a calcium fortified coffee beverage.

### Food Ingredients

Compositions of the present disclosure may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Functional Foods

Compositions of the disclosure may take the form of functional foods.

As used herein, the term “functional food” means food which is capable of providing not only a nutritional effect but is also capable of delivering a further beneficial effect to the consumer.

Accordingly, functional foods are ordinary foods that have components or ingredients (such as those described herein) incorporated into them that impart to the food a specific function—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

### Medical Foods

Compositions of the present disclosure may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

Further exemplary implementations are set forth in the following numbered clause which in no case shall be construed as limiting.

1. A method for reducing body weight in a subject, the method comprising administering to the subject an effective amount of a composition comprising dileucine.
2. The method of clause 1, wherein the dileucine is present from about 10% to 90% (w/w).
3. The method of clause 2, wherein the dileucine is present from about 30% to 70% (w/w).
4. The method of clause 3, wherein the dileucine is present at about 50% (w/w).
5. The method of clause 1, wherein the composition further comprises leucine.
6. The method of clause 1, wherein the administration of the composition to the subject synergistically increases the plasma levels of leucine relative to administration of a composition comprising leucine without dileucine.
7. The method of clause 1, wherein the subject is overweight.
8. The method of clause 7, wherein the subject is obese.
9. The method of clause 7, wherein in the composition is administered in a therapeutically effective amount.
10. The method of any preceding clause wherein the composition is a dietary supplement.
11. The method of clause 10, wherein the dietary supplement is a powder or capsule.
12. The method of clause 1, wherein the composition is in the form of a functional food.
13. The method of clause 12, wherein the functional food is a beverage, nutrition bar, yoghurt, or cereal.
14. A method for reducing body weight and/or body fat in a subject, the method comprising administering to the subject an effective amount of a composition comprising at least one amino acid or peptide chosen from: dileucine, trileucine, and Leu-Leu-R, wherein R is an amino acid or an amino acid derivative, and pharmaceutically acceptable salts thereof.
15. The method of clause 14, wherein the at least one amino acid or peptide comprises leucine and di-leucine.
16. The method of clause 15, wherein the dileucine is present from about 10% to 90% (w/w).
17. The method of clause 16, wherein the dileucine is present from about 30% to 70% (w/w).
18. The method of clause 17, wherein the dileucine is present at about 50% (w/w).
19. The method of clause 15, wherein the administration of the composition to the subject synergistically increases the plasma levels of leucine relative to administration of a composition comprising leucine without dileucine.
20. The method of clause 15, wherein the administration of the composition to the subject synergistically reduces body weight and/or body fat relative to administration of a composition comprising leucine without dileucine.
21. A method of managing a body weight disorder in a subject in need thereof comprising administering to the subject a therapeutically effective amount of a composition comprising dileucine.
22. The method of clause 21, wherein the composition further comprises leucine.
23. The method of clause 22, wherein the dileucine is present from about 10% to 90% (w/w) of the combined weight of leucine and dileucine.
24. The method of clause 23, wherein the dileucine is present from about 30% to 70% (w/w) of the combined weight of leucine and dileucine.
25. The method of clause 24, wherein the dileucine is present at about 50% (w/w) of the combined weight of leucine and dileucine.
26. The method of clause 21, wherein the body weight disorder is one or more of obesity, lipodystrophy, diabetes or metabolic syndrome, fibrosis and cancer.
27. The method of clause 26, wherein the composition is administered in conjunction with calorie restriction of the subject.
28. The method of clause 27, wherein the combined administration of the composition and calorie restriction results in greater weight loss in the subject than the weight loss produced by calorie restriction alone.
29. A method for reducing body weight in a subject, the method comprising administering to the subject an effective amount of a composition comprising dileucine.
30. The method of clause 29, wherein the dileucine is present from about 10% to 90% (w/w).
31. The method of clause 30, wherein the dileucine is present from about 30% to 70% (w/w).
32. The method of clause 31, wherein the dileucine is present at about 50% (w/w).
33. The method of clause 32, wherein the composition further comprises leucine.
33. The method of clause 29-33, wherein the administration of the composition to the subject synergistically increases the plasma levels of leucine relative to administration of a composition comprising leucine without dileucine.
34. The method of clause 29-34, wherein the subject is overweight.
35. The method of clause 34, wherein the subject is obese.
36. The method of any preceding clause, wherein the composition further comprises one or more of caffeine, green tea extract, L-carnitine, Garcinia cambogia (hydroxycitric acid), capsaicin, ginseng, taurine, silk peptides and/or octacosanol and wherein fat loss is achieved through enhancing lipolysis in the subject.
37. The method of any preceding wherein the composition further comprises one or more of fenugreek, glucomannan, Gymnema sylvestre, 5-HTP, Caralluma fimbriata, green tea extract, conjugated linoleic acid, Garcinia cambogia, and/or Yerba mate and wherein fat loss is promoted through suppression of appetite in the subject.
38. The method of any preceding clause, wherein fat loss is achieved through enhancing lipolysis in the subject.
39. The method of any preceding wherein the composition further comprises one or more of caffeine, green tea, capsaicin, Garcinia cambogia, yohimbine and bitter orange and wherein fat loss is achieved through enhancing thermogenesis in the subject.
40. A method for weight management in a subject in need there of comprising administering to the subject a composition comprising and effective amount of dileucine and further comprising and effective amount of paraxanthine.
41. The method of clause 40, wherein paraxanthine is administered in an amount of from 50 mg to about 800 mg.
42. The method of clause 41, wherein paraxanthine is administered in an amount of from 100 mg to about 400 mg.
43. The method of clause 40, wherein the administration of the composition to the subject synergistically reduces body weight and/or body fat relative to administration of a composition comprising dileucine without paraxanthine or paraxanthine without dileucine.
44. The method of any proceeding clause wherein the composition further comprises one or more of, L-Hydroxyproline, Paraxanthine, L-Tyrosine, L-Arginine, HICA, BAIBA, L-Citruline, CaHMB, Creatine monohydrate, Vitamin D3, and ATP.

### EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example I

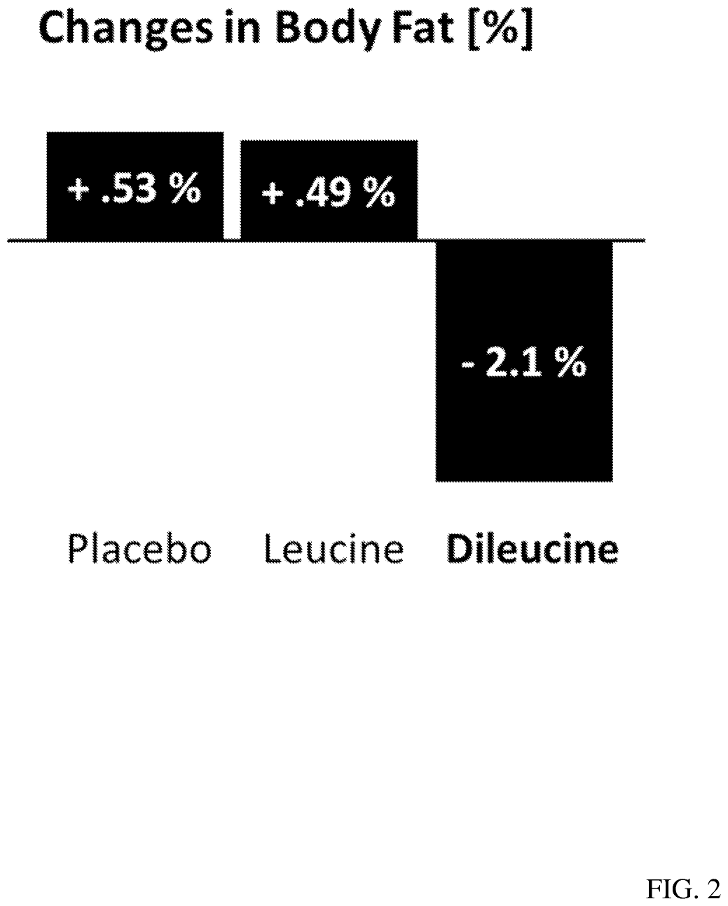

A randomized, double-blind, cross over design, resistance exercise training study was preformed to assess the impact of administration of the instantly disclosed compositions on subject body composition and body fat. Subjects were “overweight”, BMI of 26.8 in the leucine group, 25.0 in the dileucine group. Treatment groups received either leucine (2 g per day), dileucine (2 g per day), or placebo (n=7, per treatment group). Body measurements were taken at the beginning and conclusion of the study. As can be seen in FIG. 2, the dileucine treatment group showed decreased body fat following resistance training while the placebo group and leucine treatment group each showed modest increases in body fat. Taken together, these data indicate that administration of dileucine is effective in reducing body fat.

### Example II

Animal studies were conducted to investigate the effects of test substances (treatment) on weight management in male Swiss albino mice. Animals were divided into 17 groups with each group consisting of 8 animals. The test groups were treated with the test substances daily for 28 days. All the animals were subjected to treadmill training for 5 days a week for 28 days. Measurements were taken on day 0 and 28th day. The results are shown in Table 2 below.

TABLE 1

| | | | Dose | |
|---|---|---|---|---|
| | Group | | Volume | No. of |
| Groups | Description | Dose Level | (ml/kg) | animals |
| G1 | Control | Demineralized | 10 | 8 |
| | | water | | |
| G2 | Dileucine | 411 mg/kg | 10 | 8 |
| G3 | Dileucine + | 411 mg/kg + | 10 | 8 |
| | Paraxanthine | 20.55 mg/kg | | |
| G4 | Dileucine + | 411 mg/kg + | 10 | 8 |
| | CaHMB | 411 mg/kg | | |
| G5 | Dileucine + | 411 mg/kg + | 10 | 8 |
| | L-Tyrosine | 102.75 mg/kg | | |
| G6 | Dileucine + | 411 mg/kg + | 10 | 8 |
| | L-Arginine | 616.55 mg/kg | | |
| G7 | Dileucine + | 411 mg/kg + | 10 | 8 |
| | HICA | 411 mg/kg | | |
| G8 | Dileucine + | 411 mg/kg + | 10 | 8 |
| | Creatine | 411 mg/kg | | |
| | monohydrate | | | |
| G9 | Dileucine + | 411 mg/kg + | 10 | 8 |
| | Vitamin D3 | 205.5 IU | | |
| G10 | Dileucine + | 411 mg/kg + | 10 | 8 |
| | ATP | 82.2 mg/kg | | |
| G11 | Dileucine + | 411 mg/kg + | 10 | 8 |
| | BAIBA | 102.75 mg/kg | | |
| G12 | Dileucine + | 411 mg/kg + | 10 | 8 |
| | L-Citrulline | 616.5 mg/kg | | |

TABLE 2

| | Weight | Percent Difference |
|---|---|---|
| Treatment | in grams | from Placebo |
| Control | 34.15 | |
| Dileucine | 33.23 | −2.69 |
| Dileucine + Paraxanthine | 32.29 | −5.45 |
| Dileucine + L-Tyrosine | 32.58 | −4.60 |
| Dileucine + Creatine monohydrate | 32.38 | −5.18 |
| Dileucine + Vitamin D3 | 32.96 | −3.48 |
| Dileucine + ATP | 32.91 | −3.63 |
| Dileucine + CaHMB | 32.65 | −4.39 |
| Dileucine + L-Arginine | 32.63 | −4.45 |
| Dileucine + HICA | 32.94 | −3.54 |
| Dileucine + BAIBA | 32.74 | −4.13 |
| Dileucine + L-Citrulline | 32.76 | −4.07 |

Although the disclosure has been described with reference to preferred embodiments, persons skilled in the art will recognize that changes may be made in form and detail without departing from the spirit and scope of the disclosed apparatus, systems and methods.

What is claimed is:

1. A method for reducing body fat or improving body composition in a subject, the method comprising administering to the subject an effective amount of a composition comprising dileucine.

2. The method of claim 1, wherein the composition is administered such that the subject receives a daily dose of dileucine of from about 25 mg to about 5,000 mg.

3. The method of claim 2, wherein the composition is administered such that the subject receives a daily dose of dileucine of from about 500 mg to about 2,000 mg.

4. The method of claim 2, wherein the composition is administered such that the subject receives a daily dose of dileucine of from about 100 mg to about 3,000 mg.

5. The method of claim 1, wherein the composition further comprises leucine and wherein the administration of the composition to the subject synergistically increases the plasma levels of leucine relative to administration of a composition comprising leucine without dileucine.

6. The method of claim 1, wherein the composition further comprises one or more of caffeine, green tea extract, L-carnitine, Garcinia cambogia (hydroxycitric acid), capsaicin, ginseng, taurine, silk peptides and/or octacosanol and wherein fat loss is achieved through enhancing lipolysis in the subject.

7. The method of claim 1, wherein the composition further comprises one or more of caffeine, green tea, capsaicin, Garcinia cambogia, yohimbine and bitter orange and wherein fat loss is achieved through enhancing thermogenesis in the subject.

8. The method of claim 1, wherein the composition further comprises one or more of fenugreek, glucomannan, Gymnema sylvestre, 5-HTP, Caralluma fimbriata, green tea extract, conjugated linoleic acid, Garcinia cambogia, and/or Yerba mate and wherein fat loss is promoted through suppression of appetite in the subject.

9. The method of claim 1, wherein in composition is administered in conjunction with the subject's participation in a strength training regime.

10. The method of claim 1, wherein the composition is a dietary supplement, beverage, nutrition bar, yoghurt, or cereal.

14. A method for reducing body weight and/or body fat in a subject, the method comprising administering to the subject an effective amount of a composition comprising at least one amino acid or peptide chosen from: dileucine, trileucine, and Leu-Leu-R, wherein R is an amino acid or an amino acid derivative, and pharmaceutically acceptable salts thereof.

15. The method of claim 14, wherein the at least one amino acid or peptide comprises leucine and di-leucine.

16. The method of claim 15, wherein the dileucine is present from about 10% to 90% (w/w).

17. The method of claim 16, wherein the dileucine is present from about 30% to 70% (w/w).

18. The method of claim 15, wherein the administration of the composition to the subject synergistically increases the plasma levels of leucine relative to administration of a composition comprising leucine without dileucine.

19. The method of claim 15, wherein the administration of the composition to the subject synergistically reduces body weight and/or body fat relative to administration of a composition comprising leucine without dileucine.

20. A method for weight management in a subject in need there of comprising administering to the subject a composition comprising and effective amount of dileucine and further comprising and effective amount of paraxanthine.
